(19) United States
(12) Patent Application Publication
(10) Pub. No.: US 2024/0077410 A1
(43) Pub. Date: Mar. 7, 2024

(54) A METHOD AND SOFTWARE PRODUCT FOR PROVIDING A GEOMETRIC ABUNDANCE INDEX OF A TARGET FEATURE FOR SPECTRAL DATA

(71) Applicant: Xspectre AB, Stockholm (SE)

(72) Inventor: Thomas GUMBRICHT, Stockholm (SE)

(21) Appl. No.: 18/272,619

(22) PCT Filed: Jan. 21, 2022
(86) PCT No.: PCT/SE2022/050066

§ 371 Date: Jul. 17, 2023

(30) Foreign Application Priority Data
Jan. 25, 2021 (SE) 2150075-6

(51) Int. Cl. G01N 21/25; G01N 33/24; G06V 10/77; G06V 20/10; G06V 20/13

(52) U.S. Cl. CPC G01N 21/25; G01N 33/24; G06V 10/7715; G06V 20/13; G06V 20/188

(57) ABSTRACT

The present disclosure relates to a computer implemented method for providing a geometric abundance index of a target feature for spectral data and other multiband electromagnetic signals. The method (300) comprises obtaining (310) target feature spectra (110) indicative of the target feature; obtaining (320) at least one spectral endmember (103,104); determining (330) a transformation matrix (420) arranged to transform spectral data (410) to a transformed feature space (100), and transforming said target feature spectra (110) and said at least one spectral endmember (103,104) utilizing said determined transformation matrix (420); defining (340) a target feature reference line (111) in the transformed feature space (100) based on the transformed target feature spectra (110); determining (350) a coverage index (241) and a similarity index (242) in a geometric model domain based on the target feature reference line (111), wherein the coverage index (241) and the similarity index (242) are trigonometrically defined to be orthogonally oriented at all local points in the geometric model domain; and determining (360) a geometric abundance index (243) indicative of the amount of target feature based on the coverage index (241) and the similarity index (242).

(FIG. 4)

### TECHNICAL FIELD

The present disclosure relates to classifying and visualizing spectral data.

### BACKGROUND

Spectral data and other multiband electromagnetic signals are used within many fields of science, manufacturing, exploration and monitoring, including for example astronomical spectroscopy, X-ray photoelectron spectroscopy, Raman spectroscopy, fluorescence spectroscopy, nuclear magnetic resonance spectroscopy, absorption and emission spectroscopy. Electromagnetic multiband data is also commonly captured as 2D images, for instance using sensors mounted on drones, aeroplanes or satellites. The proposed method can be applied to any of these data sources, but the example used to explain the method relates to spectral satellite imagery.

Reflectance data retrieved from satellite-based sensors are used by for instance mineral explorers to identify potential mining sites, agronomists and practitioners to map crop growth and biophysical conditions at Earth's surface. Such data is particularly useful in precision agriculture, because they provide estimates of current field conditions over large areas at high spatial resolution, and is often used as an example.

Estimating crop yield is an important measurement in agronomy as it is indicative of the cumulative effects of environmental factors and management practices over the growing season. Previous work indicate the potential of remote sensing to forecast and reconstruct time series of crop yield. Systematic biases and model transferability in both time and over different regions remain obstacles, and need to be addressed by designing and testing new algorithms.

One of the most widely used remote sensing approaches is to apply statistical techniques relating one or multiple vegetation indices (VIs) to crop biomass reference data. The most commonly used vegetation indices, such as the simple ratio (SR) or normalized difference vegetation index (NDVI), are derived from visible red and near infrared (NIR) reflectance.

More advanced data mining techniques to process crop biomass reference data, such as principal components analysis and machine learning, have emerged as the efficiency of computers and availability of spectral information has increased.

### SUMMARY

One object of the invention is to provide a geometric abundance index for spectral data.

This has in accordance with the present disclosure been achieved by means of a computer implemented method for providing a geometric abundance index of a target feature for spectral data and other multiband electromagnetic signals, the method comprising the steps of

obtaining target feature spectra indicative of the target feature;

obtaining at least one spectral endmember;

determining a transformation matrix arranged to transform spectral data to a transformed feature space based on at least two spectra, wherein said at least two spectra comprises the at least one spectral endmember and/or the target feature spectra, and transforming said target feature spectra and said at least one spectral endmember utilizing said determined transformation matrix, whereby the transformed at least one spectral endmember and/or the transformed target feature spectra defines a set of feature vectors in the transformed feature space;

defining a target feature reference line in the transformed feature space based on the transformed target feature spectra, and defining a geometric model domain in the transformed feature space based on the transformed target feature spectra and the set of feature vectors, wherein said geometric model domain is an at least two-dimensional subset of the transformed feature space;

determining a coverage index and a similarity index in the geometric model domain based on the target feature reference line, wherein the value of the similarity index along the target feature reference line is constant, and wherein the coverage index and the similarity index are trigonometrically defined to be orthogonally oriented at all local points in the geometric model domain; and

determining a geometric abundance index indicative of the amount of target feature based on the coverage index and the similarity index.

This has the advantage of providing a geometric abundance index of a target feature for improved visualization and classification of spectra in the geometric model domain. This further has the advantage of providing a geometric abundance index based on both a coverage index indicative of a coverage over a background material, and a similarity index indicative of the similarity between the non-background material spectra and the target feature spectra.

A further advantage of a geometric abundance index based on a geometric solution defined from the target feature coverage and similarity is allowing a 2D or 3D visualization that is intelligible for domain experts as the transformed spectra may be based on pre-defined (domain-specific) features. A further advantage is that the method allows determining the abundance of a target feature without a linear unmixing that requires more spectral bands than the number of constituents comprising the sample material, thus providing a non-linear unmixing model for solving abundance in spectral data for a specific target feature.

The determined geometric abundance index may be related to a geometric abundance model (GAM) for estimating target feature abundances from spectral data. The GAM is based on similar basic principles as linear mixed-effect model (LMM) and principal component analysis (PCA) in that a transformation matrix is defined from a set of features with known spectral properties followed by a transformation of the spectral bands to a feature defined space. A characterizing feature of GAM is the construction of a geometric abundance index in the transformed space. The transformation matrix is defined as an orthogonalisation from a set of features with known spectral properties.

In some examples of the method, the geometric abundance index is determined in a geometric abundance realm, wherein said geometric abundance realm is defined as all points in the geometric model domain where the geometric abundance index value is above a threshold value, such as zero.

This has the advantage of improving visualization and classification of spectra transformed to the geometric model domain as the geometric abundance realm is indicative of the region of the geometric model domain wherein the abundance of the target feature is above a threshold value, such as zero. Defining a geometric abundance realm further has the advantage of allowing visual feedback during calibration as the shape of the outer boundary of the geometric abundance realm is indicative of the region where the geometric abundance index indicate presence of the target feature.

In some examples the method further comprises obtaining spectral data, and determining the geometric abundance index further comprises transforming the obtained spectral data utilizing the transformation matrix to the transformed feature space, and providing a classification and/or presentation of said transformed spectral data based on a corresponding geometric abundance index value and/or a corresponding position of the transformed spectral data in the geometric abundance realm.

In some of these examples presentation of said transformed spectral data comprises presenting the geometric model domain comprising at least the transformed spectral data, the target feature reference line and/or the geometric abundance realm.

In some examples the method further comprises obtaining spectral data, wherein obtaining at least one spectral endmember comprises obtaining at least one spectral endmember based on the spectral data obtained from the step of obtaining spectral data.

This has the advantage of providing a classification of obtained spectral data utilizing said determined geometric abundance index. This further allows determination of a geometric abundance index value for any spectra of the appropriate type, such as scoring parts of an aerial photo based on the determined abundance of a specific crop in said parts.

In some examples the method is arranged to transform spectra comprising two input bands into a 2-dimensional (2D) geometric model domain, and/or arranged to transform spectra comprising three input bands into a 3D geometric model domain.

This has the advantage of allowing a visualization and/or classification of spectra in 2D or 3D geometric model domains even for spectra with a relatively low number of input bands.

In some examples of the method, determining the geometric abundance index is based on a Euclidean deviation of the coverage index and the similarity index from a determined point of maximum target feature abundance in the geometric abundance realm.

This has the advantage of allowing a geometric abundance index to be defined such that either the coverage index value or the similarity index value may deviate such that the geometric abundance index value is reduced below zero, in other words the contribution from the coverage index value cannot compensate for the contribution from the similarity index value decreasing the geometric abundance index value below zero. An advantage of defining the geometric abundance index in such a manner allows for adjustments to the similarity index or coverage index to result in predictable changes to the geometric abundance index.

In some examples of the method, determining the geometric abundance index is further based on a determined point of null target feature abundance along the tangent of the target feature reference line. The point of null target feature abundance may be a point beyond which an experienced user determines no abundance exists, a point indicative of spectra of a frequently occurring material with spectra akin to the target feature spectra but that is not the target feature, or a point automatically determined based on the target feature reference line.

This has the advantage of allowing spectra of a known other material that typically would be incorrectly assigned an above zero target feature abundance to be used for a zero geometric abundance index constraint, thereby forcing the coverage index and/or similarity index to be adjusted such that the geometric abundance index value is zero at the point of the transformed spectra of said known other material.

In some examples the method further comprises calibrating at least one of the at least one spectral endmember, the target feature reference line, the point of maximum target feature abundance, the point of null target feature abundance along the tangent of the feature reference line, the coverage index, a convergence point of the isolines for the similarity index, and the similarity index by iteratively adjusting the corresponding value(s) and performing the corresponding steps of the method until at least one calibration criteria is fulfilled.

This has the advantage of allowing the method to be automatically or manually calibrated to improve the determined geometric abundance index so as to provide a better visualization or classification of spectral data. The method allows calibrating values, points and/or lines defining a set of constraints for the similarity index and coverage index; the similarity index and coverage index values; and how the similarity index and coverage index define the geometric abundance index.

A further advantage is that coverage index and similarity index may be calibrated by applying any regressor, thereby allowing for full use of Machine Learning capabilities while retaining user transparency.

In some of these examples calibrating comprises obtaining at least one training spectra indicative of materials with known abundance of the target feature, wherein calibrating is based on the at least one position of the transformed training spectra in the geometric model domain.

This has the advantage of allowing the geometric abundance index to be refined based on known training spectra indicative of a high target feature abundance and a low target feature abundance respectively.

In some examples of the method, obtaining the target feature spectra comprises obtaining at least one conflict reference spectra indicative of at least one feature conflicting with the target feature, wherein the method further comprises adjusting the similarity index and/or the coverage index based on the conflict reference spectra transformed utilizing the transformation matrix.

In some of these examples the similarity index and/or the coverage index is adjusted such that the point corresponding to transformed conflict reference spectra has a geometric abundance index value of zero or below.

This has the advantage of allowing the method to adjust the similarity index and/or the coverage index based on the transformed conflict reference spectra indicative of at least one feature conflicting with the target feature such that undesired overlap in the geometric model domain may be avoided.

In some examples the method comprises performing a reverse matrix transformation with one or more feature vectors omitted, thereby generating an unmixed data set.

This has the advantage of allowing improved visualization of spectra transformed to the geometric model domain and allows visualization of the original data, whether a spectral image or a spectral intensity graph, to be displayed with one or more features unmixed, such as an image of the Earth's surface with vegetation removed.

The present disclosure further relates to a computer program product comprising a non-transitory computer-readable storage medium having thereon a computer program comprising program instructions, the computer program being loadable into a processor and configured to cause the processor to perform the method for providing a geometric abundance index of a target feature for spectral data and other multiband electromagnetic signals according to any one of the preceding claims.

### BRIEF DESCRIPTION OF THE DRAWINGS

FIG. 1 depicts an illustration of transformed feature space comprising a target feature reference line.

FIG. 2a-i illustrate isolines for indices in a geometric model domain

FIG. 3 shows schematically a computer implemented method for determining a geometric abundance index of a target feature in spectral data.

FIG. 4 depicts schematically transforming spectral data into a geometric model domain in transformed feature space.

FIG. 5 depicts schematically a data processing unit comprising a computer program product.

### DETAILED DESCRIPTION

Throughout the figures, same reference numerals refer to same parts, concepts, and/or elements. Consequently, what will be said regarding a reference numeral in one figure applies equally well to the same reference numeral in other figures unless not explicitly stated otherwise. The same reference numerals will, unless stated otherwise, be used for information in real-space and transformation space, such as spectral data and the corresponding transformed spectral data in a transformed feature space.

Throughout the figure descriptions examples typically relate to classifying spectral data indicative of a photo of Earth's surface, such as an aerial or satellite photo, in relation to a specific crop and a set of background materials. In most such examples the target feature is a specific crop of interest, such as corn, and background materials are soil and/or photosynthetic vegetation. Note that the invention is not limited to classifying spectral data indicative of a photo of Earth's surface, and the invention may be used for any type of multiband electromagnetic signal data for which a spectra can be determined for a target feature and a set of background materials.

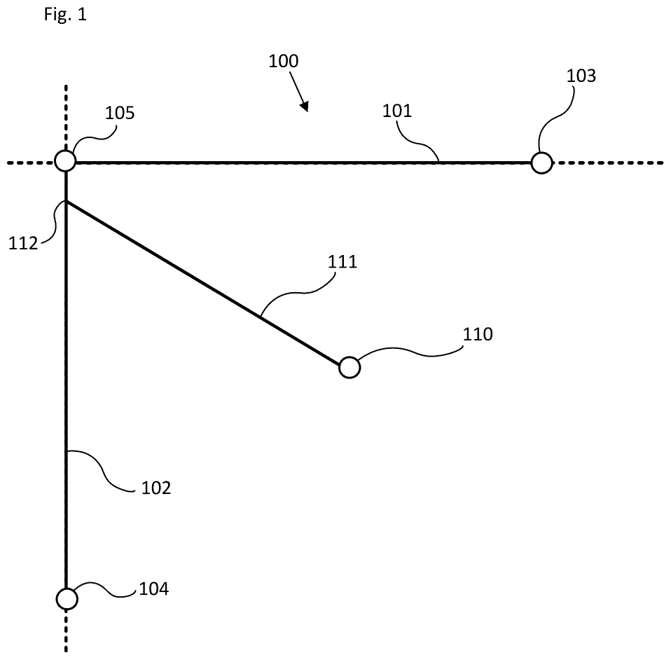

FIG. 1 depicts an illustration of transformed feature space 100. The transformed feature space 100 being a space where spectral data is transformed into via a transformation matrix. The axes of the orthogonal transformed feature space 100 are determined by at least one transformed spectral endmember 103,104 indicative of a background material, such as soil, and/or a transformed spectra of a target feature 110 indicative of the target feature, such as a specific crop of interest. In FIG. 1 the example transformed feature space 100 is defined by two transformed spectral endmembers 103,104, such as one spectral endmember indicative of soil and one spectral endmember indicative of photosynthetic vegetation.

Spectral data may be transformed to the transformed feature space 100 utilizing a transformation matrix, wherein the transformation matrix is determined based on the set of spectral endmembers 103,104 and/or the spectra of a target feature 110. Spectral data may be transformed to the transformed feature space 100 utilizing a matrix resembling said transformation matrix or any standard method that would yield a transformation to the transformed feature space 100 that is similar to utilizing said transformation matrix. The transformation matrix may be a unitary matrix. The transformation matrix is in a preferred embodiment a unitary matrix. As an example, the transformation matrix may be constructed through an orthogonalisation algorithm such as the Gram-Schmidt transformation.

In some examples the method comprises defining and utilizing a unitary matrix for the transformation to the transformed feature space, whereby the defined eigenvectors are forced to be defined as sequentially perpendicular. The set of feature vectors is in a preferred embodiment a set of orthogonal feature vectors.

This has the advantage of forcing the axis of the geometric model domain to represent orthogonally defined features.

In the example transformed feature space 100 depicted in FIG. 1 the first axis of transformed feature space is defined by a first feature vector 101 starting at a point 105 corresponding to “no signal” and going to the transformed first spectral endmember 103. The second axis is defined by a second feature vector 102 based on the transformed second spectral endmember 104. The first and/or second spectral endmember 103,104 may be indicative of a background material, such as soil or non-crop biomass.

The transformed spectra of the target feature 110 defines a target feature reference line 111 representing the transition from one or more background materials to the target feature 110 itself. The intercept 112 between the target feature reference line 111 and the second feature vector 102 corresponds to the abundance of the target feature being zero.

Moving along the target feature reference line 111 from said intercept 112 towards the transformed spectra of the target feature 110 the abundance of target feature increases until a point of maximum abundance of the target feature. Typically the point corresponding to the transformed target feature spectra 110 is at least initially the same as the point of maximum abundance of the target feature. Continuing in the trajectory of the target feature reference line 111 on the far side of the point of maximum target feature abundance decreases the abundance of target feature which may be seen as representing mixtures of the target feature with other materials. The position along the target feature reference line 111 is indicative of the coverage of the target feature over one or more background materials, such the amount of a specific crop of interest covering an area of soil.

The part of the transformed feature space 100 for which at least some values are defined is defined as a geometric model domain. The target feature reference line 111 is comprised in the geometric model domain. In the example in FIG. 1 the transformed feature space 100 is 2-dimensional and the geometric model domain is a plane extending over at least part of the transformed feature space 100. The geometric model domain is an at least two-dimensional subset of the transformed feature space 100. In some embodiments the transformed feature space 100 is at least three-dimensional, and the geometric model domain is an at least three-dimensional subset of the transformed feature space 100.

In some embodiments the tangent of the target feature reference line 111 is expressed as a line in a XY-plane defined by the first and second feature vectors 101,102, such as Y=A·X+B, wherein A is the slope of the line and B is the intercept 112 with the Y-axis.

In some embodiments of the method the first and second feature vectors 101,102 of a 2-dimensional transformed feature space 100 are defined by one transformed spectral endmember 103,104 and the transformed target feature spectra 110. Defining an axis by the transformed target feature spectra 110 allows a 2-dimensional transformed feature space 100 to be defined with only one spectral endmember 103,104.

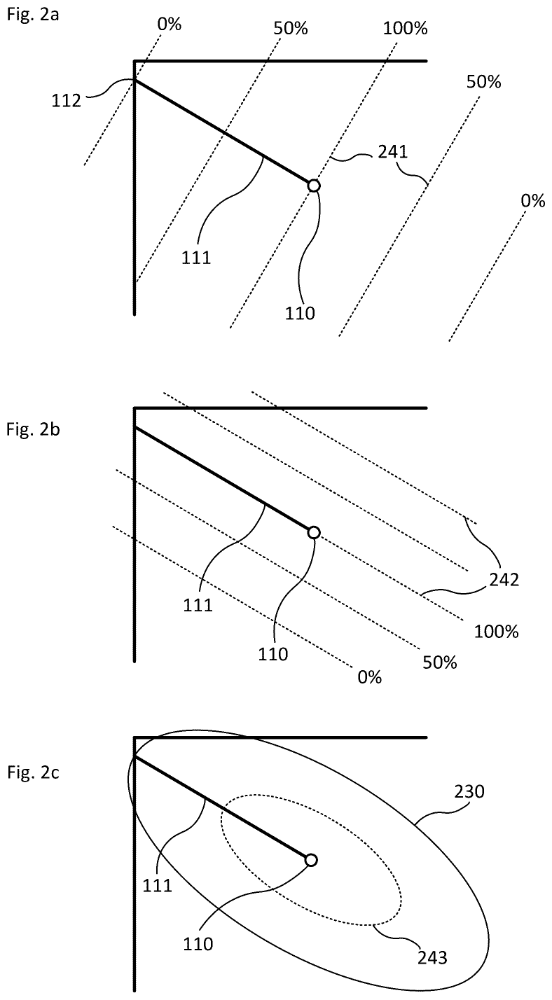

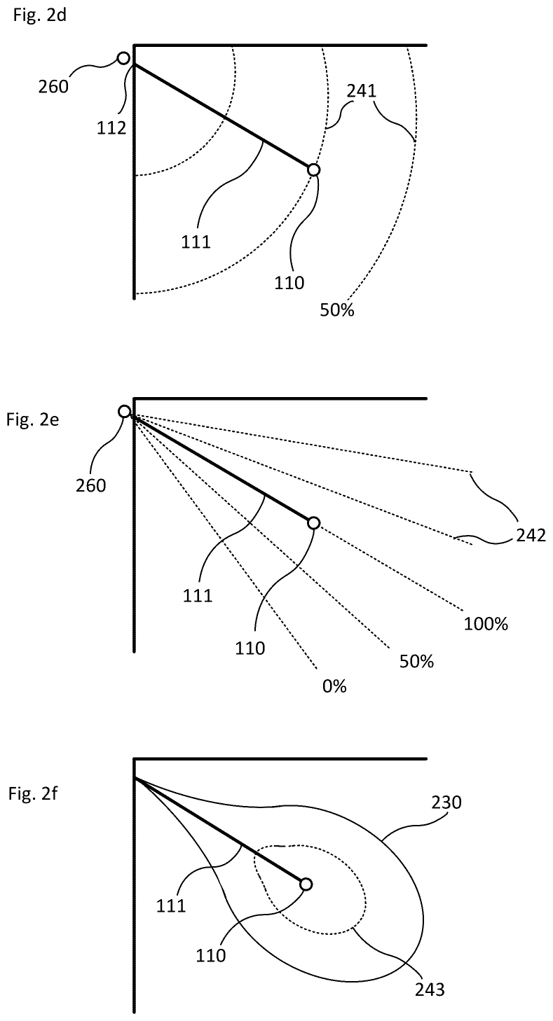

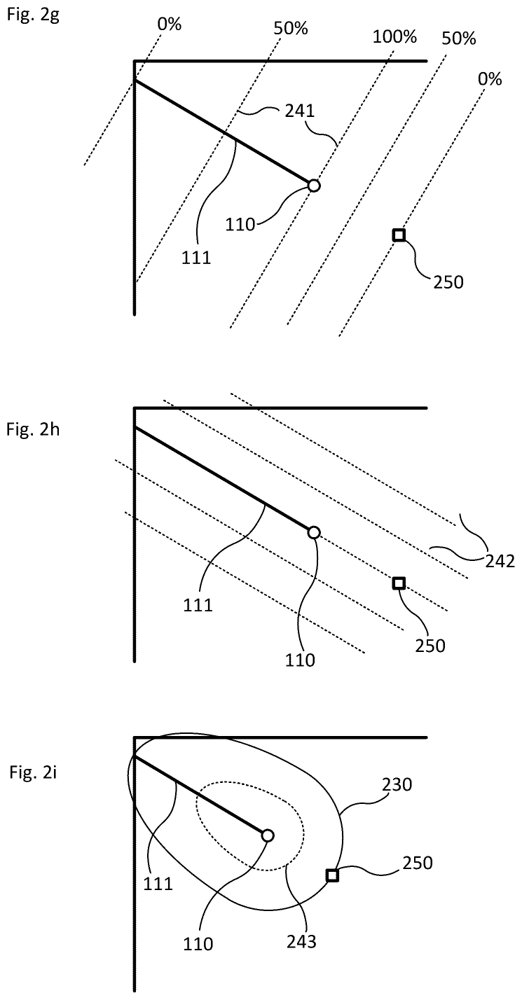

FIG. 2a-i illustrates coverage index 241, similarity index 242 and geometric abundance index 243 in the transformed feature space. The defined region of transformed feature space is called the geometric model domain. In general it may be useful to utilize a scalar field in a coordinate system for classifying a point, such as an index describing some relationship between points in the geometric model domain and the transformed spectra of the target feature 110. In these examples coverage index 241, similarity index 242 and geometric abundance index 243 have been defined based on the transformed spectra of the target feature 110 and corresponding target feature reference line 111 such as described in FIG. 1. In these examples the point of the transformed spectra of the target feature 110 in the transformed feature space is the point of maximum abundance of the target feature. In these examples the same reference number are used for the indices 241,242,243 and the corresponding index isolines 241,242,243.

In these examples the tangent of the target feature reference line 111 may be expressed as a line in an XY-plane defined by a first and a second feature vector, such as Y=A·X+B, wherein A is the slope of the line and B is the intercept 112 with the Y-axis. In FIG. 2a-i the geometric model domain is illustrated with such an XY-plane in the plane of each figure.

The coverage index 241, the similarity index 242 and the geometric abundance index 243 are scalar fields defined in at least part of the geometric model domain, and in FIG. 2a-i these indices 241,242,243 are visualized by some of their corresponding isolines. The indices 241,242,243 and correspondingly the isolines are typically dependent, such as the coverage index 241 and the similarity index 242 isolines being orthogonal in the geometric model domain. The illustrations of the isolines in FIG. 2a-i aim to provide an approximate shape in order to visualize the indices 241,242,243, however, note that said illustrated isolines may fail to mathematically match the stated dependencies or listed equations.

In the examples of FIG. 2a-i both the coverage index 241 and the similarity index 242 are mathematically defined such that the numerical value of said indices 241,242 at the point of maximum abundance of the target feature equals zero. Deviations from the point of maximum target feature abundance causes the absolute values of the coverage index 241 and/or the similarity index 242 to increase. This has the additional advantage of allowing identification of negative and positive sides of both the coverage 241 and similarity index 242. In these examples the geometric abundance index 243 is defined from a maximum geometric index value from which the square root of the summed squares of the coverage and similarity indices is subtracted, as will be described in Eq.6.

FIG. 2a illustrates a geometric model domain of transformed feature space comprising a transformed spectra of a target feature 110, a corresponding target feature reference line 111 and isolines of a coverage index 241. The coverage index 241 value represents the amount of coverage over a background material, such as the amount of biomass coverage over soil. In the example illustrated in FIG. 2a the isoline of the coverage index 241 at the point of the transformed target feature spectra 110 is substantially orthogonal to the target feature reference line 111 and points along said isoline have the same coverage index value as the point of maximum abundance of the target feature, as indicated by 100%. The isoline of the coverage index 241 going through the point of intercept 112 of the target feature reference line 111 has a coverage index value indicative of zero coverage, as indicated by 0%. In this example the coverage index isolines 241 are substantially parallel.

The coverage index 241, COV, may be based on a Euclidian distance from a point (X, Y) to a convergence point (X₀,Y₀), according to Eq.1 and Eq.2, wherein COVₘₐₓ is the straight line distance from the point of maximum abundance of the target feature (X_{TFmax}, Y_{TFmax}) to the convergence point (X₀,Y₀), whereby the coverage index value for the point of maximum abundance of the target feature is zero.

COV=COVₘₐₓ−\sqrt{(X−X₀)²+(Y−Y₀)²} (Eq.1)

COVₘₐₓ=\sqrt{(X_{TFmax}−X₀)²+(Y_{TFmax}−Y₀)²} (Eq.2)

FIG. 2b illustrates a geometric model domain of transformed feature space comprising the transformed spectra of the target feature 110, the corresponding target feature reference line 111 and isolines of a similarity index 242. The similarity index 242 represents the similarity with the target feature. In the example illustrated in FIG. 2b the similarity index 242 corresponds to the coverage index 241 in FIG. 2a. The isoline of the similarity index 242 going though the point of maximum abundance of the target feature represent the highest similarity, as indicated by 100%. In this example the isolines of the similarity index 242 are substantially parallel to the target feature reference line 111.

The similarity index 241, SIM, may have isolines converging at a point (X₀,Y₀) and having the highest similarity along a line Y=A·X+B wherein the similarity index along said line has a numerical value of zero. Eq.3-5 define an example similarity index 241 for a point (X, Y) wherein the point of convergence (X₀,Y₀) is a point along said line, and wherein the similarity index value is scaled by a scaling factor S. The point of convergence may be the same as the convergence point used to define the coverage index. In this example a similarity index value is calculated as the rotational angle of the perpendicular deviation compared to the target feature reference 111 line multiplied with the SIM scaling factor, S. S may be defined such that similarity index 242 is scaled numerically equal to coverage index 241 at the point of maximum abundance of the target feature. In this example the orthogonal geometric distance that indicates zero abundance due to similarity equals the geometric distance from the point of maximum abundance to the point of zero abundance along the target feature reference line. Adjusting these geometric distances to be more similar typically results in a more circular geometric abundance realm. Increasing S further widens the geometric abundance realm orthogonally from the target feature reference line and decreasing it narrows the same realm. FIG. 2a-c illustrate a narrow geometric abundance realm, having a shorter distance from the point of maximum target feature abundance to the zero isolines for SIM than for COV, corresponding to lower values of S. The perpendicular deviation of the point (X,Y) from the target feature reference line 111 may be calculated by first finding the point (X_{tfrl}, Y_{tfrl}) along the target reference line 111 with a coverage index value equal to the coverage index value of said point (X,Y), as seen in Eq.4 and Eq.5.

$$SIM = S \times arc cos ( 1 - \frac{{( X - {X}_{tfrl} )}^{2} + {( Y - {Y}_{tfrl} )}^{2}}{2 \times ( {( X - {X}_{0} )}^{2} + {( Y - {Y}_{0} )}^{2} )} ) \qquad ( Eq . 3 )$$

$${X}_{tfrl} = {X}_{0} + \frac{\sqrt{{( X - {X}_{0} )}^{2} + {( Y - {Y}_{0} )}^{2}}}{\sqrt{1 + {A}^{2}}} \qquad ( Eq . 4 )$$

$${Y}_{tfrl} = {X}_{tfrl} \times A + B \qquad ( Eq . 5 )$$

The example in FIG. 2b with similarity index 242 substantially parallel isolines around the target feature reference line 111 corresponds to a point of convergence (X₀,Y₀) located far away.

The constructional basis of the similarity index 242 with substantially parallel isolines in FIG. 2b resembles that of the Perpendicular Vegetation Index (PVI) used as a proxy for estimating vegetation density from Earth observing imagery.

The coverage index 241 and similarity index 242 are trigonometrically defined to be orthogonally oriented at all local points in the geometric model domain, thereby the isolines for the coverage index 241 and similarity index 242 are orthogonal in all points of the geometric model domain.

In some examples the isolines of the similarity index 242 converges in a point in the transformed feature space 100. In these examples, each isoline of the coverage index 241 is at the same distance from said point of convergence. Said point of convergence may be the convergence point (X₀,Y₀) in Eq.1-4.

Returning to FIG. 2a-b and considering points along the target feature reference line 111. The coverage index 241 values changes along the target feature reference line 111. The similarity index 242 values stay the same along the target feature reference line 111, as illustrated in FIG. 2b by the isoline parallel to the target feature reference line 111.

The coverage index 241 values for points along the target feature reference line 111 represent a varying amounts of non-background covering the background, such as amounts of biomass covering soil. The similarity index 242 values for points along the target feature reference line 111 represent that the non-background material covering the background is “pure” target feature, such as the biomass covering the soil being a specific crop of interest and not containing other vegetation or crop types. Thus, the coverage index 241 and similarity index 242 values for points along the target feature reference line 111 together describe that the background is covered by varying amounts of “pure” target feature, such as fields with substantially only soil and varying amounts of a specific crop.

Points along a coverage index 241 isoline, with a coverage index value corresponding to a point along the target feature reference line 111, represent an amount coverage of the background with varying mixtures of the target feature and/or other materials, wherein the amount of coverage is comparable to said point along the target feature reference line 111.

Interpreting and classifying spectral data transformed to points along the target feature reference line 111 may be relatively straightforward. However, points further away from the target feature reference line 111 may be less intuitive and more complicated to interpret.

FIG. 2c illustrates a geometric model domain of transformed feature space comprising the transformed spectra of the target feature 110, the corresponding target feature reference line 111, an isoline of a geometric abundance index 243 and a corresponding geometric abundance realm 230. The geometric abundance index 243 represented by said isoline and the geometric abundance realm 230 in FIG. 2c corresponds to the coverage index 241 and the similarity index 242 represented in FIGS. 2a and 2b respectively.

The geometric abundance index 243 is based on the coverage index 241 and the similarity index 242. In the example in FIG. 2c the geometric abundance index 243 is based on a Euclidean deviation of the coverage index 241 and the similarity index 242 from a determined point of maximum target feature abundance in the geometric model domain. In this example said determined point of maximum target feature abundance is the same point as the point of the transformed target feature spectra 110.

GAI=GAIₘₐₓ−\sqrt{(ΔCOV)²+(ΔSIM)²} (Eq.6)

Eq.6 describes an example equation defining the geometric abundance index 243, GAI, wherein GAIₘₐₓ is the geometric abundance index 243 value at the point of maximum target feature abundance, ΔCOV is the difference in coverage index 241 value for a point compared to the point of maximum target feature abundance, and ΔSIM is the difference in similarity index 242 value for said point compared to the point of maximum target feature abundance. The point of maximum target feature abundance is typically the same as the point corresponding to the transformed target feature spectra 110, but the point of maximum target feature abundance may be selected as another point, for example due to calibration or user preference. In the examples wherein COV and SIM are defined as zero for the point of maximum target feature abundance, then ΔCOV and ΔSIM in Eq.6 become COV and SIM.

The geometric abundance realm 230 is the part of the geometric model domain wherein the geometric abundance index 243 is above a threshold value, such as zero. In FIG. 2c the point of the transformed target feature spectra 110 has the maximum geometric abundance value, points along the depicted geometric abundance index isoline 243 has 50% of the maximum geometric abundance value and points along the circumference of the geometric abundance realm 230 has geometric abundance index value of zero. Adjusting the value of GAIₘₐₓ in Eq.6, or alternatively adjusting the threshold value, may change the size of the geometric abundance realm 230.

FIGS. 2c, 2f and 2i each illustrate an example geometric abundance index 243 defined such that a point with coverage index value and similarity index value representing the maximum coverage and zero similarity has zero geometric abundance index value, and also index values representing maximum similarity and zero coverage has zero geometric abundance index value. In other words, in these examples geometric abundance realms 230 with threshold values of zero will at least not extend beyond the area enclosed isolines representing zero coverage and zero similarity. This allows the coverage index 241 and/or the similarity index 242 to be adjusted to e.g. handle a constraint point indicative of zero geometric abundance index value.

The geometric abundance index 243 provides a value for transformed spectral data indicative of the abundance of the target feature as estimated from the spectral data. In some examples spectral data transformed to the geometric model domain is presented visually and the corresponding points of transformed spectral data may be classified based on their positional relationship to the transformed spectra of the target feature 110, the target feature reference line 111, any isolines of the geometric abundance index 243 and/or the border of the geometric abundance realm 230. In some of these examples the spectral data comprises sample spectra and reference spectra indicative of known materials, whereby the visually presented geometric model domain further allows the user to classify a point of transformed sample spectra based on a point of transformed reference spectra with known properties.

The geometric abundance realm 230, and its abundance index 243, the coverage index 241 and/or the similarity index 242 may further be visualized with a gradient (not shown) representing respective indices 241,242,243.

The coverage index 241, the similarity index 242, the point of maximum target feature abundance and/or the target feature reference line 111 may be adjusted and calibrated to provide a geometric abundance index 243 that better represents the known properties of the reference spectra. Similarly the equations based on coverage index 241 and the similarity index 242 that determines the geometric abundance index 243 may be redefined in order to better classify and visualize the transformed spectral data.

FIG. 2d-i illustrates two examples of how the geometric abundance index 243 may change based on changes to the coverage index 241 and the similarity index 242. Unless otherwise stated the geometric abundance index 243 is defined based on the coverage index 241 and the similarity index 242 in the same manner in FIG. 2d-f and FIG. 2g-i as in FIG. 2a-c, such as defined by Eq.6.

FIG. 2d-f illustrate coverage index 241, similarity index 242 and geometric abundance index 243 in the transformed feature space, wherein the isolines for the coverage index 241 are curved and the isolines for the similarity index 242 are not substantially parallel.

FIG. 2d illustrates an example coverage index 241 in a geometric model domain of the transformed feature space corresponding to the similarity index in FIG. 2e. In this example the coverage index 241 relates to the distance from a point of convergence 260 located along the tangent of the target feature reference line 111, wherein points with coverage index 241 representing the highest coverage are at the same distance from said point of convergence 260 as the point of the transformed target feature spectra 110.

It is to be understood that the values of the coverage index 241 are not limited to being linearly dependent on the distance from the point of convergence 260 located along the tangent of the target feature reference line 111. For example, there can be a difference in radius between the isoline for maximum abundance, 100%, and each of the two half-maximum abundance isolines, 50%, such that the rate of maximum change in coverage index 241 is different in different parts of the geometric model domain.

FIG. 2e illustrates an example geometric model domain of the transformed feature space wherein the similarity index 242 has isolines converging at the point of convergence 260.

The similarity index 242 and the coverage index 241 being trigonometrically defined to be orthogonally oriented at all local points in the geometric model domain causes a similarity index 242 with isolines converging at a convergence point 260 to have a corresponding coverage index 241 with isolines being parallel circle arcs around said convergence point 260.

FIG. 2f illustrate the geometric abundance index 243 in an example geometric model domain of the transformed feature space corresponding to the coverage index 241 in FIG. 2d and the similarity index 242 in FIG. 2e. Compared to the geometric abundance index 243 in FIG. 2c, the geometric abundance index 243 in FIG. 2f has a significant difference in the rate of change in the value of the geometric abundance index 243 in directions perpendicular to the target feature reference line 111, between points along the target feature reference line 111.

FIGS. 2a-c and 2d-f represent the end-points of a continuum of possible convergences, ranging from a point of convergence 260 of substantially parallel isolines located very far away from the target feature reference line 111 depicted in FIG. 2a-c to a point of convergence 260 very close to, or coinciding with, the target feature reference line 111 depicted in FIG. 2d-f. The point of convergence 260 may thus be used for adjusting the coverage, similarity and/or geometric abundance indices 241,242,243.

FIG. 2g-i illustrate coverage index 241, similarity index 242 and geometric abundance index 243 in the transformed feature space, wherein a conflict deforms the coverage index 241. The deformation based on conflict(s) may cause the trigonometric orthogonal relation between coverage and similarity to deviate.

FIG. 2g illustrates the coverage index 241 in an example geometric model domain of the transformed feature space based on the example described for FIG. 2a further comprising a constraint point 250. The constraint point 250 represents a point of null target feature abundance, thus the geometric abundance index 243 in said point should ideally not be a positive value. The constraint point 250 may be indicative of a transformed conflict reference spectra, such as a spectral endmember of a frequently occurring material that is known to risk being mistaken for the target feature. The constraint point 250 may be introduced into the geometric model domain automatically based on algorithms and any suitable machine learning method for analysing spectral data and/or manually based on user experience.

In FIG. 2g the coverage index 241 in FIG. 2a has been compressed for points beyond the isoline 241 through the point of the transformed target feature spectra 110. In this example the adjustment to the coverage index 241 makes the conflict point 250 have a coverage index 241 value such that the geometric abundance index 243 value becomes exactly zero at said conflict point 250. In this example the conflict point 250 has the same similarity index 242 value as the point of maximum target feature abundance and has a coverage index 241 value representing zero coverage.

Note that FIG. 2g-i illustrates a special case where the conflict point 250 is on the similarity index 242 isoline representing the highest similarity whereby a coverage index value representing zero coverage may be required in order to obtain a geometric abundance index 243 value of zero or lower for the conflict point 250. If the conflict point 250 was located outside the similarity index 242 isoline representing the highest similarity, then the coverage index 241 may be adjusted such that the coverage index value of the conflict point 250 represents some amount of coverage and the geometric abundance index 243 value of the conflict point 250 is zero or below.

Any number of conflicting conflict points 250 may be included, and the adjustments to the coverage index 241 and coverage index 242 may be solved by linear interpolation, spline, and/or inverse distance weights, or any other established interpolation method.

FIG. 2h illustrates the similarity index 242 corresponding to the coverage index 241 in FIG. 2g. The similarity index in FIG. 2h may be identical to the similarity index in FIG. 2b as the shape of the coverage index isolines 241 are the same in FIGS. 2a and 2g and only the rate of change in coverage index values are different in some parts of the geometric model domain.

FIG. 2i illustrates the geometric abundance index 243 corresponding to the coverage index 241 and similarity index 242 of FIG. 2g-h. Compared to FIG. 2c the geometric abundance realm 230 in FIG. 2i is compressed on the far side of the point of the transformed target feature spectra 110 due to the compressed corresponding coverage index 241. In this example the geometric abundance index 243 is defined such that the conflict point 250 has a geometric abundance index value of zero. In this example the deformation of the coverage index 241 has allowed deviations from the local orthogonal conditions between coverage and similarity. Any deformation adjustment of the coverage index 241 and/or the similarity index 242 may be set to either allow such deviations, or be adjusted to maintain the local orthogonality (or a combination thereof).

FIG. 2g-i illustrates a situation where a conflict deforms the coverage index 241. It is to be understood that the similarity index 242 may also be deformed asymmetrically with respect to the tangent of the target feature line 111. It is to be understood that both the coverage index 241 and/or similarity index 242 may be deformed by one or more conflicts or restricting conditions.

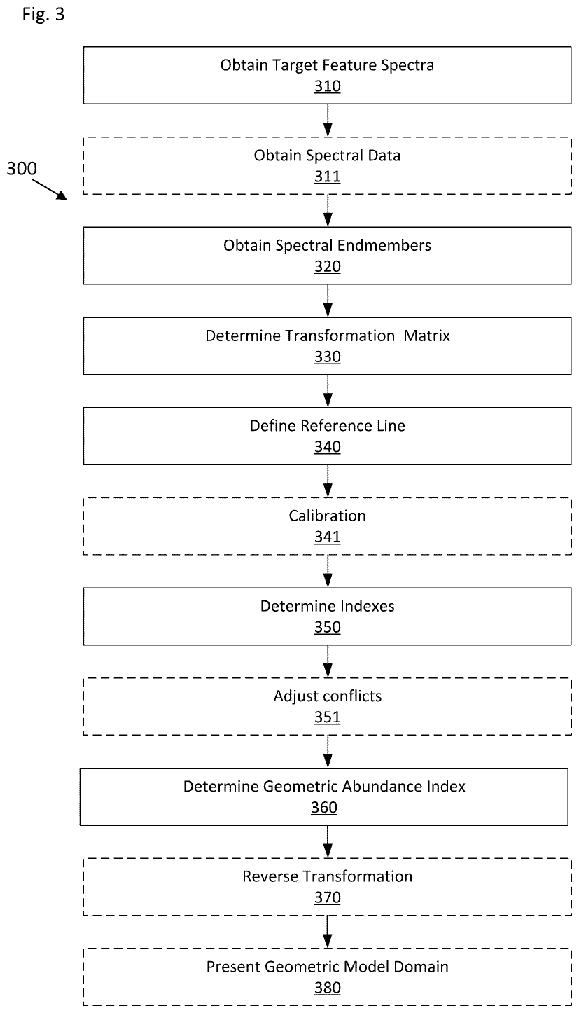

FIG. 3 shows schematically a computer implemented method for providing a geometric abundance index of a target feature for spectral data and other multiband electromagnetic signals. The method 300 comprises the steps of
obtaining 310 target feature spectra indicative of the target feature;
obtaining 320 at least one spectral endmember;
determining 330 a transformation matrix arranged to transform spectral data to a transformed feature space based on at least two spectra, wherein said at least two spectra comprises the at least one spectral endmember and/or the target feature spectra, and transforming said target feature spectra and said at least one spectral endmember utilizing said determined transformation matrix, whereby the transformed at least one spectral endmember and/or the transformed target feature spectra defines a set of feature vectors in the transformed feature space;
defining 340 a target feature reference line in the transformed feature space based on the transformed target feature spectra, and defining a geometric model domain in the transformed feature space based on the transformed target feature spectra and the set of feature vectors;
determining 350 a coverage index and a similarity index in the geometric model domain based on the target feature reference line, wherein the value of the similarity index along the target feature reference line is constant, and wherein the coverage index and the similarity index are trigonometrically defined to be orthogonally oriented at all local points in the geometric model domain; and
determining 360 a geometric abundance index based on the coverage index and the similarity index in a geometric abundance realm, wherein the geometric abundance realm is defined as all points in the geometric model domain where the geometric abundance index value is above a threshold value, such as zero.

The example method performs a stepwise conversion of electromagnetic signals to a geometric abundance index, as illustrated in FIG. 3. The initial steps of obtaining 310 target feature spectra, obtaining 311 spectral data, obtaining 320 spectral endmembers, and determining 330 the transformation matrix, may each be accomplished utilizing well known practices. The method 300 may be applied to spectral data captured with any technique and to differently defined transformation matrices. Utilizing a unitary matrix as the transformation matrix for the linear transformation of signal data to the transformed feature space, has several advantages. The application uses the unitary matrix in preferred embodiments, but it should be understood that the transformation from signal data to feature space may be accomplished also with any other rectangular array representing signal data.

The expression “obtaining target feature spectra indicative of the target feature” relates to for example, obtaining the target feature's spectra from a spectral database or from a sample or a picture element with an a priori known abundance of target feature.

The expression “obtaining at least one spectral endmember” relates to for example, obtaining the endmember's spectra from a spectral database or a pure endmember sample or from a picture element representing the pure endmember.

It is to be understood that the expression “determining a transformation matrix arranged to transform spectral data to a transformed feature space” relates to determining an orthogonalisation for the transformation matrix arranged to transform spectral data.

In some embodiments the set of feature vectors is a set of orthogonal feature vectors.

The transformation matrix and the transformed feature space may be defined from two spectral endmembers, of which one may be the target feature itself. The transformation matrix and the transformed feature space may be defined by three or more spectral endmembers. In some examples with only two spectral endmembers the transformed feature space is 2-dimensional, while examples with at least three spectral endmembers the transformed feature space may be 3-dimensional and so forth. A starting point of the geometric model domain (X=0, Y=0, Z=0 in a 3D transformed feature space) may be defined by either using one spectral endmember as an offset prior to the transformation matrix transformation, or using “no signal” as the offset. In those examples using a spectral endmember as an offset, a 2-dimensional geometric model domain requires at least three spectral endmembers, and in those examples a 3-dimensional geometric model domain requires at least four spectral endmembers and so forth. Note that in these examples the target feature spectra may be used as one of the spectral endmembers.

Determining 330 a transformation matrix, and transforming said target feature spectra and said at least one spectral endmember utilizing said determined transformation matrix is not limited to determining and utilizing a matrix for transformation. Several alternatives exist for transforming spectral data to a transformed feature space that yield similar results as utilizing a matrix, in this example the preferred embodiment is to determine and utilize a unitary matrix. In some embodiments determining 330 a transformation matrix, and transforming utilizing said determined transformation matrix comprises determining a standard transformation method for transforming spectral data to the target feature space and utilizing said determined standard transformation method. In some embodiments determining 330 a transformation matrix, and transforming utilizing said determined transformation matrix comprises determining and utilizing a unitary matrix.

It is to be understood that a step utilizing a transformation matrix that is a unitary matrix also relates to utilizing a matrix that has a minor numerical deviation compared to a corresponding unitary matrix.

The term target feature spectra 110 refers to a spectra indicative of a target feature for which a geometric abundance index 243 is sought. The target feature may for example be a specific crop and the corresponding target feature spectra may be based on an aerial photo of said crop. The same reference number is used for the target feature spectra 110 and the transformed target feature spectra 110. For example, the target feature spectra 110 may be target feature's spectra from a spectral database or from a sample or a picture element with an a priori known abundance of target feature.

The term spectral endmember 103,104 is indicative of the spectral properties of pure or well defined feature. A spectral endmember 103,104 may for example be indicative of a background material, such as soil. The same reference number is used for spectral endmembers 103,104 and the transformed spectral endmembers 103,104. For example, the spectral endmember 103,104 may be the endmember's spectra from a spectral database or from a pure endmember sample or a picture element representing the pure endmember

While the target feature spectra 110 may be considered a spectral endmember of the target feature, herein, unless otherwise stated, the term spectral endmember 103,104 is not used for the target feature spectra 110. Unless otherwise stated the term spectral endmember 103,104 relates to spectra other than the target feature spectra 110.

The term transformation matrix 420 refers to a matrix arranged to transform spectra to a transformed feature space 100. In some preferred examples the transformation matrix 420 is a unitary matrix. In some examples the eigen-vectors defining a transformation matrix 420 are defined based on spectral libraries comprising predetermined spectra and/or directly from spectra extracted from obtained spectral data, such as photographs or spectrometers.

It is to be understood that a step utilizing a transformation matrix 420 that is a unitary matrix also relates to utilizing a matrix that has a minor numerical deviation compared to a corresponding matrix that fulfils the criteria for being a unitary matrix.

The term transformed feature space 100 refers to the space into which the transformation matrix 420 transforms spectra and in which the target feature reference line 111 and the indices 241,242,243 are defined. The transformed feature space 100 being defined by a set of feature vectors 101,102, wherein the set of feature vectors are based on at least one transformed spectral endmember 103,104 and/or the transformed target feature spectra 110.

The term set of feature vectors 101,102 refers to a plurality of vectors based on at least two spectra consisting of at least one transformed spectral endmember 103,104 and/or the transformed target feature spectra 110. In some examples with a set of feature vectors consisting of two feature vectors, a first feature vector may define an X-axis in a transformed feature space, and a second feature vector may define an orthogonal Y-axis in said transformed feature space. In some of these examples the geometric model domain may be defined in said XY-plane.

The term geometric model domain refers to the defined region of the transformed feature space 100. The geometric model domain is an at least two-dimensional subset of the transformed feature space 100. In some examples the transformed feature space is the part of the transformed feature space for which the similarity index and/or the coverage index has a defined value. In some examples the transformed feature space is 3-dimensional (X-Y-Z) and the geometric model domain may be defined as a 2-dimensional plane in said transformed feature space (e.g. X-Y, X-Z and/or Y-Z). The geometric model domain comprises an area or volume in the transformed feature space 100 containing the location of the transformed target feature spectra 110 and the target feature reference line 111. Typically, the transformed spectral endmembers define the axis of the transformed feature space.

The term target feature reference line 111 refers to a line in the transformed feature space based on the transformed target feature spectra. The target feature reference line is comprised in the geometric model domain. The target feature reference line defines at least locally the coverage index 241 and the similarity index 242. The similarity index value for points along the target feature reference line is the maximum similarity index value, thus the tangent of the target feature reference line defines the isoline for the maximum similarity value. In 2-dimensional and/or 3D geometric model domains the target feature reference line is defined as a straight line. Typically, the target feature reference line 111 is defined based on the transformed target feature spectra 110 and the set of feature vectors 101,102, such that the maximum abundance of the target feature coincides with position of the transformed target feature spectra 110 and equals zero at the point where the target feature reference line 111 crosses one of the set of feature vectors 101,102.

The term coverage index 241 refers to a scalar field in the geometric model domain indicative of an amount of coverage of a background material, such as biomass covering soil. In some examples the coverage index value of a point is based on a distance from a point of convergence, whereby isolines for coverage index values become circle arcs around said convergence point.

The term similarity index 242 refers to a scalar field in the geometric model domain indicative of the similarity between non-background material spectra and the transformed target feature spectra, such as the similarity between spectra of biomass and the target feature. The isoline for the maximum similarity index value is the tangent of the target feature reference line. In some examples the isolines for similarity index values converge in a point. The coverage index and the similarity index are trigonometrically defined to be orthogonally oriented at all local points in the geometric model domain. The local orthogonality can be allowed to deviate when adjusting either similarity or coverage index for a conflicting feature.

The term geometric abundance index 243 refers to a scalar field in the geometric model domain indicative of the distance from a point of maximum abundance in the two dimensions formed by the coverage index and similarity index. The geometric abundance index is the geometric proximity from the point of maximum abundance to the, linear or non-linear, combined differences of the coverage and similarity indices compared to said point of maximum abundance. In some examples the geometric abundance index is based on a Euclidean deviation of the coverage index and the similarity index from a determined point of maximum target feature abundance in the geometric model domain, such as defined by Eq.6. Typically, the geometric abundance index 243 is the based on the Euclidean distance from a point of maximum abundance of the target feature, which prior to any calibration is set equal to the target feature spectra 110, wherein the Euclidean distance is calculated from the values of the coverage index 241 and the similarity index 242.

The same reference number is used for the coverage, similarity and geometric abundance indices 241,242,243 and their corresponding isolines.

The term geometric abundance realm 230 refers to a region of the geometric model domain defined by all points with a geometric abundance index value above a threshold value, such as zero. In some examples visualization of the geometric abundance realm comprises adding a visual gradient of the geometric abundance index value in the geometric abundance realm.

In some embodiments of the method, at least one of the at least one spectral endmember is indicative of a background material. In some of these examples the at least one spectral endmember is indicative of a background material.

In some embodiments the method, obtaining 320 at least one spectral endmember comprises obtaining at least two spectral endmembers. In some of these examples determining 330 a transformation matrix is based on said at least two spectral endmembers.

In some embodiments the method, obtaining 320 at least one spectral endmember comprises obtaining at least three spectral endmembers. In some of these examples determining 330 a transformation matrix is based on said at least three spectral endmembers.

In some embodiments the method further comprises obtaining 311 spectral data, and determining 360 the geometric abundance index further comprises transforming the obtained spectral data with the transformation matrix to the transformed feature space, and providing a classification and/or presentation of said transformed spectral data based on a corresponding geometric abundance index value and/or a corresponding position of the transformed spectral data in the geometric abundance realm. In some of these some embodiments obtaining 320 at least one spectral endmember comprises obtaining at least one spectral endmember based on the spectral data obtained from the step of obtaining 311 spectral data.

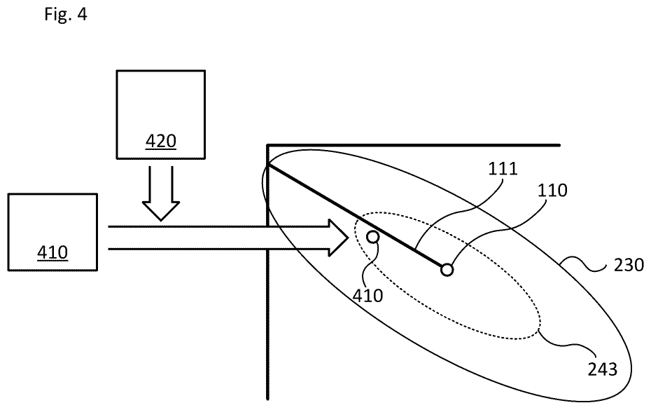

In some of these embodiments presentation of said transformed spectral data comprises presenting the geometric model domain comprising at least the transformed spectral data, the target feature reference line and/or the geometric abundance realm. In some of these embodiments the geometric model domain is two dimensional, whereby the presentation becomes a two dimensional image, such as FIGS. 1 and 2a-i depicting a 2D geometric model domain and with FIG. 4 also depicting transformed spectral data.

In some embodiments presentation of said transformed spectral data comprises visualizing the transformed spectral data and the geometric abundance realm.

In some embodiments, the method, instead of obtaining 320 at least one spectral endmember, determining 330 a transformation matrix, defining 340 a target feature reference line, determining 350 a coverage index and a similarity index, and determining 360 a geometric abundance index, comprises steps of obtaining a pre-determined geometric abundance index, and obtaining a pre-determined transformation matrix. These embodiments further comprise obtaining 311 spectral data, and transforming the obtained spectral data with the obtained transformation matrix to the transformed feature space, and providing a classification of said transformed spectral data based on the obtained geometric abundance index, such as a geometric abundance index value. In some of these embodiments the method further comprises presenting the geometric model domain comprising visualizations indicative of said transformed spectral data, the target feature reference line and/or the geometric abundance realm.

In some embodiments determining 360 the geometric abundance index is based on a Euclidean deviation of the coverage index and the similarity index from a determined point of maximum target feature abundance in the geometric abundance realm.

In some embodiment determining 360 the geometric abundance index is based on a determined point of null target feature abundance in the geometric model domain.

In some embodiment determining 360 the geometric abundance index is based on a determined point of null target feature abundance along the tangent of the target feature reference line.

It is to be understood that the point of null target feature abundance may be a point beyond which an experienced user determines no abundance exists, a point indicative of spectra of a frequently occurring material with spectra akin to the target feature spectra but that is not the target feature, or a point automatically determined based on the target feature reference line and the at least one spectral endmember which define the geometric model domain.

In some embodiments the method further comprises calibrating 341 the at least one spectral endmember, the target feature reference line, the point of maximum target feature abundance, the point of null target feature abundance along the feature reference line, the coverage index, and/or the similarity index by iteratively adjusting the corresponding value(s) and performing the corresponding steps of the method 300 until at least one calibration criteria is fulfilled.

The target feature reference line may be calibrated 341 by adjusting
the point defining zero target feature abundance,
the point defining maximum target feature abundance, or
the numerical value of indices at the point of maximum target feature abundance.

In 2 dimensional and/or 3D geometric model domains the target feature reference line is defined as a straight line. In geometric model domains of at least 4-dimensions the target feature reference line may also be defined as a curved line and/or a plane.

It is to be understood that the step of calibrating 341 may occur at one or more instances while and/or after performing the other steps of the method 300. The step of calibrating 341 may comprise iterating at least one other step of the method 300.

In some embodiments the method, calibrating 341 comprises obtaining at least one training spectra indicative of materials with known abundance of the target feature, wherein calibrating 341 is based on the at least one position of the transformed training spectra in the geometric model domain.

In some embodiments of the method, calibrating 341 comprises adjusting the convergence point of the isolines for the similarity index and/or the curvature of the isolines of the coverage index.

In some embodiments of the method, calibrating 341 comprises adjusting the spacing between zero value isolines and/or the rate of change of the similarity index and/or the coverage index.

In some embodiments of the method, obtaining 310 the target feature spectra comprises obtaining at least one conflict reference spectra indicative of at least one feature conflicting with the target feature, and

the method 300 further comprises adjusting 351 the similarity index and/or the coverage index based on conflict reference spectra transformed utilizing the transformation matrix.

In some embodiments of the method, adjusting the similarity index and/or the coverage index may cause the trigonometric orthogonal relation between coverage and similarity to deviate.

It is to be understood that the expression “the trigonometric orthogonal relation between coverage and similarity to deviate” relates to the local isolines of the coverage index and the similarity index.

In some embodiments the method, further comprising translating at least some geometric abundance index values to physical units.

In some embodiments the method further comprises performing 370 a reverse matrix transformation with one or more feature vectors omitted, thereby generating an unmixed data set.

In some embodiments the method further comprises presenting 380 a visualization of the geometric model domain comprising the target feature reference line, the set of feature vectors, the transformed target feature spectra, the transformed obtained spectral data, the geometric abundance realm, at least one isoline for the coverage index, at least one isoline for the similarity index, at least one isoline for the geometric abundance index, and/or the transformed training spectra and/or conflict spectra.

FIG. 4 depicts schematically transforming spectral data into a geometric model domain in transformed feature space. In this example the depicted geometric model domain is the geometric model domain depicted in FIG. 2c and defined in the corresponding description. The geometric model domain comprises the transformed target feature spectra 110 and the corresponding target feature reference line 111. The illustration of the geometric model domain further comprises a geometric abundance index isoline 243 corresponding to half the maximum geometric abundance index value, and a geometric abundance realm 230 enclosing the points of the geometric model domain with geometric abundance index values above zero.

The spectral data 410 may be indicative of a part of a photo, such as a part of an aerial photo or a part of any other electromagnetic multiband signal. The spectral data 410 is transformed into the transformed feature space by utilizing a transformation matrix 420. The transformation matrix 420 is arranged to transform spectral data 410 to the transformed feature space, and in this example the transformation matrix 420 is determined by two spectral endmembers indicative of background material.

In the example in FIG. 4 the transformed spectral data 410 is inside the geometric abundance realm 230 and inside the geometric abundance index isoline 243. From visual inspection of the position of the transformed spectral data 410 in the geometric model domain indicates that the geometric abundance value for the spectral data 410 is more than half the maximum value, the spectral data 410 shows significantly lower coverage compared to the target feature spectra 110, and said coverage is by material that spectrally is relatively similar to the target feature spectra 110.

In this example the target feature spectra 110 may be indicative of an aerial photo of a fully developed corn field, and the spectral data 410 may be indicative of an aerial photo of a corn field in an earlier growth stage. Thus the vector between the transformed target feature spectra 110 and the position of the transformed spectral data 410 may be interpreted as the component parallel to the target feature reference line 111 corresponding to the amount soil blocked out by corn plants, and the component perpendicular to said line 111 corresponding to how close to the corn plants are to being fully developed.

In this example transforming additional spectral data 410 indicative of aerial photos of corn fields at different known levels of maturity may provide a geometric model domain comprising a path indicative of healthy corn development. Transformed sample spectral data 410 indicative of aerial photos of unknown fields may then be compared in said geometric model domain comprising the path indicative of healthy corn development.

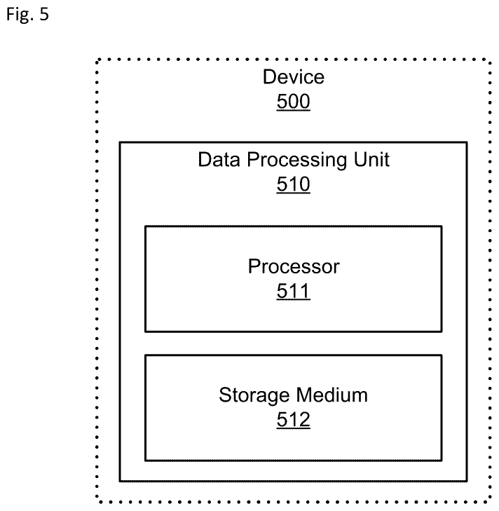

FIG. 5 depicts schematically a data processing unit 510 comprising a computer program product for providing a geometric abundance index of a target feature for spectral data. FIG. 5 depicts a data processing unit 510 comprising a computer program product comprising a non transitory computer-readable storage medium 512. The non-transitory computer-readable storage medium 512 having thereon a computer program comprising program instructions. The computer program is loadable into a data processing unit 510 and is configured to cause a processor 511 to carry out the method for providing a geometric abundance index of a target feature for spectral data in accordance with the description of FIG. 3.

The data processing unit 510 may be comprised in a device 500.

1. A computer implemented method for providing a geometric abundance index of a target feature for spectral data and other multiband electromagnetic signals, the method comprising the steps of
obtaining target feature spectra indicative of a target feature;
obtaining at least one spectral endmember;
determining a transformation matrix arranged to transform spectral data to a transformed feature space based on at least two spectra, wherein said at least two spectra comprises at least one spectral endmember and/or the target feature spectra, and transforming said target feature spectra and said at least one spectral endmember utilizing said determined transformation matrix, whereby the transformed at least one spectral endmember and/or the transformed target feature spectra defines a set of feature vectors in the transformed feature space;
wherein defining a target feature reference line in the transformed feature space based on the transformed target feature spectra, and defining a geometric model domain in the transformed feature space based on the transformed target feature spectra and the set of feature vectors, wherein said geometric model domain is an at least two-dimensional subset of the transformed feature space;
determining a coverage index and a similarity index in the geometric model domain based on the target feature reference line, wherein a value of the similarity index along the target feature reference line is constant, and wherein the coverage index and the similarity index are trigonometrically defined to be orthogonally oriented at all local points in the geometric model domain; and
determining a geometric abundance index indicative of an amount of target feature based on the coverage index and the similarity index.

2. The method according to claim 1, wherein the geometric abundance index is determined in a geometric abundance realm, and wherein said geometric abundance realm is defined as all points in the geometric model domain where the geometric abundance index value is above a threshold value, such as zero.

3. The method according to claim 1, further comprising obtaining spectral data, wherein determining the geometric abundance index further comprises transforming the obtained spectral data utilizing the transformation matrix to the transformed feature space, and providing a classification and/or presentation of said transformed spectral data based on a corresponding geometric abundance index value and/or a corresponding position of the transformed spectral data in a geometric abundance realm.

4. The method according to claim 3, wherein obtaining at least one spectral endmember comprises obtaining at least one spectral endmember based on the spectral data obtained from the step of obtaining spectral data.

5. The method according to claim 1, wherein the method is arranged to transform spectral data comprising two input bands into a 2-dimensional geometric model domain, and/or arranged to transform spectral data comprising three input bands into a 3D geometric model domain.

6. The method according to claim 1, wherein determining the geometric abundance index is based on a Euclidean deviation of the coverage index and the similarity index from a determined point of maximum target feature abundance in a geometric abundance realm.

7. The method according to claim 6, wherein determining the geometric abundance index is further based on a determined point of null target feature abundance along a tangent of the target feature reference line.

8. The method according to claim 1, further comprises calibrating at least one of the at least one spectral endmember, the target feature reference line, the point of maximum target feature abundance, the point of null target feature abundance along a tangent of the feature reference line, the coverage index, a convergence point of isolines for the similarity index, and the similarity index by iteratively adjusting the corresponding value(s) and performing the corresponding steps of the method until at least one calibration criteria is fulfilled.

9. The method according to claim 8, wherein calibrating comprises obtaining at least one training spectra indicative of materials with known abundance of the target feature, wherein calibrating is based on the at least one position of the transformed training spectra in the geometric model domain

10. The method according to claim 1, wherein obtaining the target feature spectra comprises obtaining at least one conflict reference spectra indicative of at least one feature conflicting with the target feature, and wherein the method further comprises adjusting the similarity index and/or the coverage index based on the conflict reference spectra transformed utilizing the transformation matrix.

11. The method according to claim 1, further comprising translating at least some geometric abundance index values to physical units.

12. The method according to claim 1, further comprising performing a reverse matrix transformation with one or more feature vectors omitted, thereby generating an unmixed data set.

13. A computer program product comprising a non-transitory computer-readable storage medium having thereon a computer program comprising program instructions, the computer program being loadable into a processor and configured to cause the processor to perform the method for providing a geometric abundance index of a target feature for spectral data and other multiband electromagnetic signals according to claim 1.
